(19) United States
(12) Patent Application Publication
(10) Pub. No.: US 2018/0085053 A1
(43) Pub. Date: Mar. 29, 2018

(54) MEASUREMENT DEVICE AND MEASUREMENT METHOD

(71) Applicant: SEIKO EPSON CORPORATION, Tokyo (JP)

(72) Inventors: Taki HASHIMOTO, Shiojiri-shi (JP); Tetsuo MANO, Fujimi-machi (JP)

(73) Assignee: SEIKO EPSON CORPORATION, Tokyo (JP)

(21) Appl. No.: 15/697,085

(22) Filed: Sep. 6, 2017

(30) Foreign Application Priority Data
Sep. 26, 2016 (JP) 2016-186937

(51) Int. Cl. A61B 5/00; A61B 5/024

(52) U.S. Cl. CPC A61B 5/4818; A61B 5/14551; A61B 5/7257; A61B 5/02416

(57) ABSTRACT

A measurement device includes a calculation section adapted to calculate oxygen saturation from a pulse wave signal of a test subject, and a determination section adapted to determine presence or absence of chest cavity expansion motion of the test subject from the pulse wave signal.

### BACKGROUND

### 1. Technical Field

The present invention relates to a technology for evaluating respiratory condition of a test subject.

### 2. Related Art

In the past, there has been proposed a variety of technologies for evaluating respiratory condition of a test subject. In JP-A-2016-10616 (Document 1), for example, there is disclosed a configuration of determining whether the apnea syndrome during sleep of the test subject is occlusive or central in accordance with a combination of presence or absence of the breath sound and presence or absence of a motion of the chest.

In the technology of Document 1, a sound signal is used for the determination on presence or absence of the breath sound, and an acceleration signal different from the sound signal is used for the determination on presence or absence of the motion of the chest. A respirometric sensor (a microphone) is used for generation of the sound signal, and a body motion measuring sensor (an acceleration sensor) separated from the respirometric sensor is used for generation of the acceleration signal. Therefore, the technology of Document 1 has a problem that the configuration of the device for estimating the respiratory condition becomes complicated.

### SUMMARY

An advantage of some aspects of the invention is to simplify the configuration of the measurement device for determining presence or absence of the chest cavity expansion motion of the test subject.

A measurement device according to a preferred aspect of the invention includes a calculation section adapted to calculate oxygen saturation from a pulse wave signal of a test subject, and a determination section adapted to determine presence or absence of chest cavity expansion motion of the test subject from the pulse wave signal. In this configuration described above, the pulse wave signal is commonly used for both of the calculation of the oxygen saturation and the determination on the presence or absence of the chest cavity expansion motion. Therefore, the configuration of the measurement device is simplified compared to the configuration of using a signal (e.g., a signal representing a breath sound or a signal representing a body motion), which is different from the signal used for the calculation of the oxygen saturation, for the determination on the presence or absence of the chest cavity expansion motion of the test subject. Here, the chest cavity expansion motion denotes a motion of expanding the chest cavity in accordance with the contraction of the diaphragm.

In the preferred aspect of the invention, the determination section may determine the presence or absence of the chest cavity expansion motion in a case in which the oxygen saturation drops. In this configuration described above, the presence or absence of the chest cavity expansion motion is determined in the case in which the oxygen saturation drops. Therefore, the load of the measurement device can be reduced compared to the configuration of determining the presence or absence of the chest cavity expansion motion irrespective of the value of the oxygen saturation.

In the preferred aspect of the invention, the determination section may determine that the chest cavity expansion motion is present in a case in which a peak is detected in a frequency band no lower than 0.1 Hz and no higher than 0.5 Hz out of frequency spectrum of the pulse wave signal, and determine that the chest cavity expansion motion is absent in a case in which no peak is detected in the frequency band no lower than 0.1 Hz and no higher than 0.5 Hz. In this configuration, the presence or absence of the chest cavity expansion motion is determined in accordance with the presence or absence of the peak in the frequency band no lower than 0.1 Hz and no higher than 0.5 Hz out of the frequency spectrum of the pulse wave signal. The pulse wave signal includes the frequency component due to the pulsation of the blood vessel and the frequency component due to the chest cavity expansion motion, and there is a tendency that the frequency component due to the breathing or the chest cavity expansion motion is observed in the frequency band no lower than 0.1 Hz and no higher than 0.5 Hz. According to the aspect described above, using the tendency that the frequency component due to the pulsation of the blood vessel and the frequency component due to the breathing or the chest cavity expansion motion are different in frequency from each other, it is possible to accurately determine the presence or absence of the chest cavity expansion motion.

In the preferred aspect of the invention, the determination section may determine that the chest cavity expansion motion is present in a case in which a variation period of a component value of a low frequency component of the signal is within a predetermined range, and determine that the chest cavity expansion motion is absent in a case in which the variation period of the component value of the low frequency component of the signal is out of the predetermined range. In this configuration described above, the presence or absence of the chest cavity expansion motion is determined in accordance with the variation period of the component value of the low frequency component of the pulse wave signal. There is a tendency that the component value of the low frequency component of the pulse wave signal is different in variation period between the case in which the chest cavity expansion motion is present and the case in which the chest cavity expansion motion is absent. According to the aspect described above, using the tendency that the variation period is different between the case in which the chest cavity expansion motion is present and the case in which the chest cavity expansion motion is absent, it is possible to accurately determine the presence or absence of the chest cavity expansion motion.

In the preferred aspect of the invention, the predetermined range may be a range no shorter than 2 seconds and not longer than 10 seconds. In this configuration, it is determined that the chest cavity expansion motion is present in the case in which the component value of the low frequency component of the pulse wave signal varies with the period no shorter than 2 seconds and not longer than 10 seconds. In the case in which the chest cavity expansion motion is present, the component value of the low frequency component of the pulse wave signal has a tendency of varying with the period no shorter than 2 seconds and not longer than 10 seconds. According to the aspect described above, using the tendency that the component value of the low frequency component of the pulse wave signal varies with the period no shorter than 2 seconds and not longer than 10 seconds in the case in which the chest cavity expansion motion is present, it is possible to accurately determine the presence or absence of the chest cavity expansion motion.

In the preferred aspect of the invention, in a case in which oxygen saturation drops, the determination section may determine that an obstructive sleep apnea syndrome occurs in a case in which it is determined that the chest cavity expansion motion is present, and determine that a central sleep apnea syndrome occurs in a case in which it is determined that the chest cavity expansion motion is absent. Here, in the case in which the oxygen saturation drops, there is a high possibility that the respiration of the test subject stops. In this configuration, in the case in which there is a high possibility that the respiration stops, it is possible to discriminate between the obstructive sleep apnea syndrome and the central sleep apnea syndrome in accordance with the presence or absence of the chest cavity expansion motion. Therefore, it is possible to discriminate the sleep apnea syndrome between the obstructive type and the central type in accordance with the combination of the presence or absence of the oxygen saturation drop and the presence or absence of the chest cavity expansion motion.

In the preferred aspect of the invention, the measurement device may further include a counting section adapted to count a number of times of the oxygen saturation drop. In this configuration, the number of times of the oxygen saturation drop is counted. Therefore, compared to the configuration of not counting the number of times of the oxygen saturation drop, it is possible to figure out the tendency (e.g., the frequency) of the oxygen saturation drop.

In the preferred aspect of the invention, the counting section may count the number of times of the oxygen saturation drop for each of the obstructive sleep apnea syndrome and the central sleep apnea syndrome. In this configuration, the number of times of the oxygen saturation drop is counted for each of the obstructive sleep apnea syndrome and the central sleep apnea syndrome. Therefore, compared to the configuration of counting the number of times of the oxygen saturation drop without discriminating between the obstructive sleep apnea syndrome and the central sleep apnea syndrome, it is possible to individually figure out the tendency (e.g., the frequency) of the drop of the oxygen saturation for each of the obstructive sleep apnea syndrome and the central sleep apnea syndrome.

In the preferred aspect of the invention, the measurement device may further include an identification section adapted to identify sleep depth of the test subject, and the counting section may count the number of times of the oxygen saturation drop in each of levels of the sleep depth. In this configuration, the number of times of the oxygen saturation drop is counted for each of the levels of the sleep depth. Therefore, compared to the configuration of counting the number of times of the oxygen saturation drop without discriminating between the levels of the sleep depth, it is possible to figure out the tendency (e.g., the frequency) of the drop of the oxygen saturation for each of the levels of the sleep depth.

A measurement method according to a preferred aspect of the invention includes calculating, by a computer, oxygen saturation from a pulse wave signal of a test subject, and determining, by the computer, presence or absence of a chest cavity expansion motion of the test subject. According to the method described above, substantially the same functions and the advantages as those of the measurement device according to the invention can be obtained.

### BRIEF DESCRIPTION OF THE DRAWINGS

The invention will be described with reference to the accompanying drawings, wherein like numbers reference like elements.

FIG. 1 is a side view of a measurement device according to a first embodiment of the invention.

FIG. 2 is a configuration diagram focusing attention on the function of the measurement device.

FIG. 3 is a diagram showing a temporal change of a detection signal.

FIG. 4 is a diagram showing a temporal change of a component value of a low frequency component of the detection signal in the case in which the test subject breathes normally.

FIG. 5 is a diagram showing a temporal change of a component value of a low frequency component of the detection signal in the case of assuming an obstructive sleep apnea syndrome.

FIG. 6 is a diagram showing a temporal change of a component value of a low frequency component of the detection signal in the case of assuming a central sleep apnea syndrome.

FIG. 7 is a diagram showing a temporal change of oxygen saturation.

FIG. 8 is a diagram showing the frequency spectrum of the detection signal of the case in which the chest cavity expansion motion is present.

FIG. 9 is a schematic diagram of a measurement result table.

FIG. 10 is a diagram showing a display example of the counting result.

FIG. 11 is a flowchart of a measurement process.

FIG. 12 is a flowchart of a counting process.

FIG. 13 is a configuration diagram focusing attention on the function of the measurement device according to a second embodiment of the invention.

FIG. 14 is a schematic diagram of a measurement result table.

FIG. 15 is a diagram showing a display example of the counting result.

FIG. 16 is a flowchart of a measurement process.

### DESCRIPTION OF EXEMPLARY EMBODIMENTS

### First Embodiment

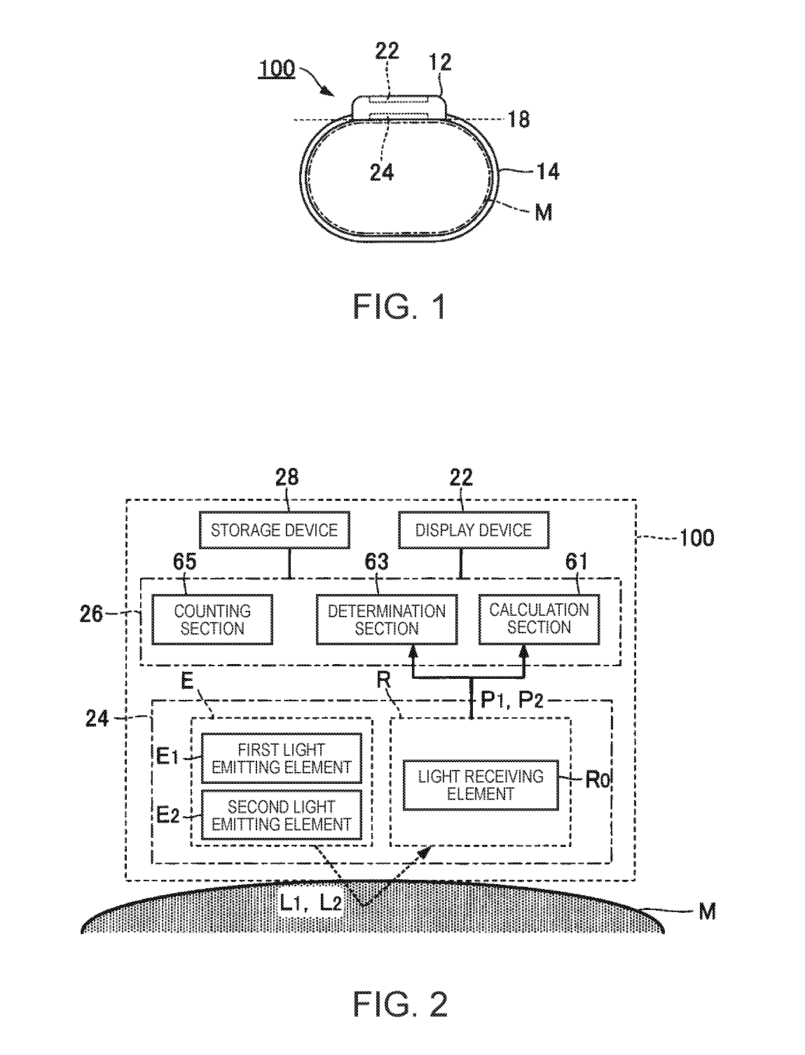

FIG. 1 is a side view of a measurement device 100 according to a first embodiment of the invention. The measurement device 100 according to the first embodiment is a measurement instrument for evaluating the respiratory condition of the test subject, and is attached to a region (hereinafter referred to as a “measurement region”) M to be a measurement target out of the body of the test subject. The measurement device 100 according to the first embodiment is wristwatch-type portable equipment provided with a housing section 12 and a belt 14, and can be attached to the wrist of the test subject by winding the belt 14 around the wrist as an illustration of the measurement region M.

The measurement device 100 according to the first embodiment evaluates the respiratory syndrome during sleep of the test subject. The respiratory syndrome is classified into an obstructive type and a central type. In the case of a normal respiratory condition, a respiration instruction is made, and thus, the chest cavity expansion motion is made to thereby achieve the intake of air to the lungs. Here, the state in which the respiration instruction is not made due to abnormality of the respiratory center, and therefore the respiration cannot be achieved is referred to as the central sleep apnea syndrome (hereinafter referred to as “CSAS”). In contrast, the state in which the chest cavity expansion motion is made, but the upper respiratory tract is blocked by narrowing of the upper respiratory tract due to a decrease in activity of the upper respiratory tract dilator, and therefore the respiration cannot be achieved is referred to as the obstructive sleep apnea syndrome (hereinafter referred to as “OSAS”).

Here, in the case in which the intake of the air to the lungs is not made (i.e., the apnea syndrome), the oxygen saturation (SpO₂) of the test subject drops. The oxygen saturation denotes the proportion (%) of the hemoglobin coupled with oxygen to the hemoglobin in the blood of the test subject, and an index for evaluating the respiratory function of the test subject. In either of the states of OSAS and CSAS, the intake of the air to the lungs is not made, and therefore, the oxygen saturation decreases. However, the chest cavity expansion motion appears in the case of OSAS, while the chest cavity expansion motion does not appear in the case of CSAS. On the assumption of the tendency described above, the measurement device 100 according to the first embodiment discriminate the apnea syndrome during sleep of the test subject between OSAS and CSAS by performing the calculation of the oxygen saturation of the test subject and the determination on presence or absence of the chest cavity expansion motion.

FIG. 2 is a configuration diagram focusing attention on the function of the measurement device 100. As illustrated in FIG. 2, the measurement device 100 according to the first embodiment is provided with a display device 22, the detection device 24, the control device 26, and the storage device 28. The control device 26 and the storage device 28 are disposed inside the housing section 12. The display device (e.g., a liquid crystal display panel) 22 is disposed on a surface (e.g., a surface on the opposite side to the measurement region M) of the housing section 12 as illustrated in FIG. 1, and displays a variety of types of images including the measurement result under the control by the control device 26.

The detection device 24 shown in FIG. 2 is a sensor module for generating a detection signal P corresponding to the state of the measurement region M, and is disposed on, for example, the opposed surface (hereinafter referred to as a “detection surface”) 18 to the measurement region M out of the housing section 12. The detection surface 18 is a flat surface or a curved surface. The detection device 24 according to the present embodiment generates a first detection signal P1 and a second detection signal P2 used for the calculation of the oxygen saturation and the determination on presence or absence of the chest cavity expansion motion. As illustrated in FIG. 2, the detection device 24 is provided with a light emitting section E and a light receiving section R. The light emitting section E and the light receiving section R are disposed on the detection surface 18 opposed to the measurement region M.

The light emitting section E shown in FIG. 2 includes a first light emitting element E1 and a second light emitting element E2, and emits light L (L1 and L2) to the measurement region M. For example, a light emitting diode (LED) is preferably used as each of the first light emitting element E1 and the second light emitting element E2. It should be noted that it is also possible for each of the first light emitting element E1 and the second light emitting element E2 to emit coherent light (i.e., a laser beam) high in coherency. As each of the light emitting elements E1, E2, there can be applied a vertical cavity surface emitting laser (VCSEL), a photonic crystal laser, a semiconductor laser, and so on. In the first embodiment, the light L1 and the light L2 are different in wavelength λ from each other. For example, the light L1 is near infrared light (wavelength λ1=800 nm through 1300 nm), and the light L2 is infrared light (wavelength λ2=600 nm through 800 nm). The first light emitting element E1 emits the light L1 during a first period, and the second light element E2 emits the light L2 during a second period. The first period and the second period are alternately repeated with a predetermined cycle on the time axis.

The light L emitted from the light emitting section E (the first light emitting element E1 and the second light emitting element E2) enters the measurement region M, and at the same time, repeats reflection and scattering inside the measurement region M, and is then emitted toward the detection surface 18 to reach the light receiving section R. Therefore, the light emitting section E and the light receiving section R function as a reflective optical sensor together with each other.

The light receiving section R includes a light receiving element R0, and generates a first detection signal P1 and a second detection signal P2 corresponding to a light reception level of the light reaching from the measurement region M. Specifically, the light receiving element R0 receives the light L1, which is emitted from the first light emitting element E1 in each of the first periods and then transmitted through the measurement region M, to generate the first detection signal P1 corresponding to the light reception level, and receives the light L2, which is emitted from the second light emitting element E2 in each of the second periods and then transmitted through the measurement region M, to generate the second detection signal P2 corresponding to the light reception level. A photoelectric conversion element such as a photodiode (PD) for receiving the light L on the light receiving surface opposed to the measurement region M is preferably used as the light receiving element R0. It should be noted that the detection device 24 includes, for example, a drive circuit for driving the light emitting section E due to the supply of a drive current, and an output circuit (e.g., an amplifier circuit and an A/D converter) for amplifying and then A/D converting the output signal of the light receiving section R, which are omitted from the illustration in FIG. 1.

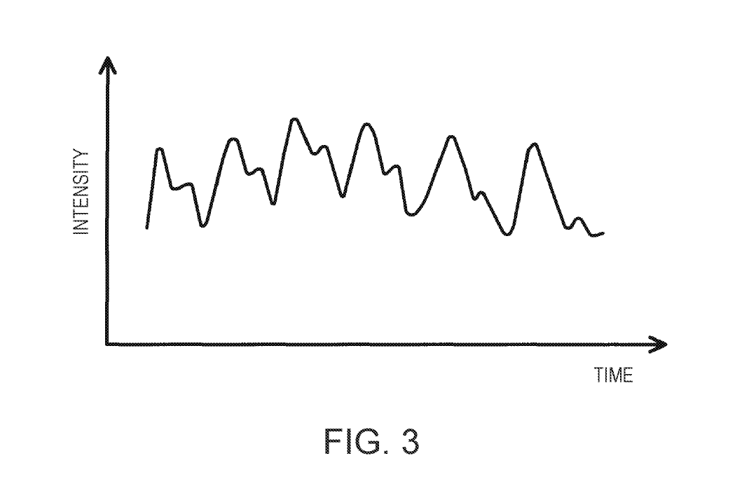

The blood vessel in the measurement region M recurrently expands and contracts with an equivalent period to the heart rate. Since the blood flow due to the blood in the blood vessel between the expansion period and the contraction period, the detection signal P (the detection signal P1 or the detection signal P2), which is generated by the light receiving section R in accordance with the light reception level from the measurement region M, is a pulse wave signal including a periodical variation component corresponding to the variation of the blood flow of an artery in the measurement region M as illustrated in FIG. 3.

The detection signal P (P1 or P2) includes a component value Q(AC) (Q1(AC) or Q2(AC)) of the high frequency component and a component value Q(DC) (Q1(DC) or Q2(DC)) of the low frequency component. The component value Q(AC) of the high frequency component is a pulse wave component periodically (period of about 1 second) varying in tandem with the pulsation of the test subject, and is extracted by a high-pass filter from the detection signal P, for example. In contrast, the component value Q(DC) of the low frequency component is a component (ideally a direct-current component kept steadily) varying with sufficiently long period (e.g., several seconds through several tens of seconds) compared to the component value Q(AC) of the high frequency component, and is extracted with a low-pass filter from the detection signal P, for example.

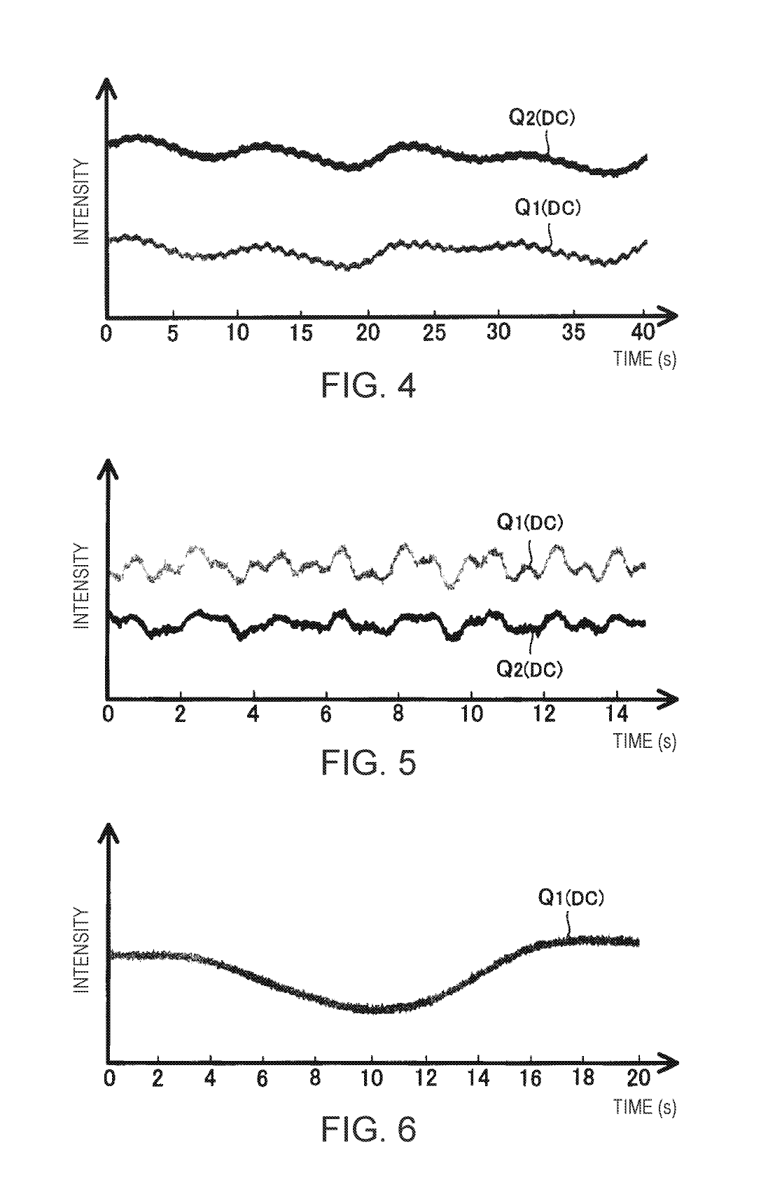

FIG. 4 through FIG. 6 are graphs of the temporal variation of the component value Q(DC) of the low frequency component respectively observed in a plurality of cases with the respiratory conditions varied from each other. Firstly, FIG. 4 shows the temporal variation of the component value Q1(DC) of the low frequency component of the first detection signal P1 and the temporal variation of the component value Q2(DC) of the low frequency component of the second detection signal P2 in the case (the chest cavity expansion motion and the intake of the air to the lungs are present) in which the test subject makes a deep breath. In other words, FIG. 4 shows the temporal variation of each of the component value Q1(DC) and the component value Q2(DC) in the case in which the test subject is breathing normally. The component value Q1(DC) and the component value Q2(DC) each vary with a period of about 10 seconds as shown in FIG. 4.

FIG. 5 shows the temporal variation of the component value Q1(DC) of the low frequency component of the first detection signal P1 and the temporal variation of the component value Q2(DC) of the low frequency component of the second detection signal P2 in the case (the chest cavity expansion motion is present, but the intake of the air to the lungs is absent) in which the test subject makes the action of expanding the chest cavity in the state in which, for example, the upper respiratory tract is blocked so as not to intake the air. In other words, FIG. 5 shows the temporal variation of each of the component value Q1(DC) and the component value Q2(DC) in the case of assuming the OSAS. The component value Q1(DC) and the component value Q2(DC) each vary with a period of about 2 seconds as shown in FIG. 5.

FIG. 6 is the temporal variation of the component value Q1(DC) of the low frequency component of the first detection signal P1 in the case (both of the chest cavity expansion motion and the intake of the air to the lungs are absent) in which the test subject makes an action of stopping the respiration. In other words, FIG. 6 shows the temporal variation of the component value Q1(DC) in the case of assuming the CSAS. As shown in FIG. 6, the component value Q1(DC) varies for about 20 seconds in accordance with increase and decrease of the blood flow due to the fact that the intake of the air to the lungs is not made. It should be noted that in FIG. 6, the variation for about 20 seconds is illustrated, but the variation of the component value Q1(DC) of the low frequency component varies between individuals. For example, the variation period longer than 10 seconds and not longer than 60 seconds is common.

As is understood from the above description, in the case of assuming the OSAS, the component value Q(DC) of the low frequency component of the detection signal P varies with the variation period (the period of about 2 seconds through 10 seconds) similar to the variation period of the component value Q(DC) of the low frequency component in the case in which the test subject is in the normal respiratory condition. In contrast, in the case of assuming the CSAS, the component value Q(DC) of the low frequency component of the detection signal P varies with the variation period (the period longer than 10 seconds and not longer than 60 seconds) sufficiently longer than the variation period of the component value Q(DC) of the low frequency component in the case in which the test subject is in the normal respiratory condition. Specifically, there is a tendency that the component value Q(DC) of the low frequency component varies with the frequency of about 2 seconds through 10 seconds in the case in which the chest cavity expansion motion is present, and varies with the frequency longer than 10 seconds and not longer than 60 seconds in the case in which the chest cavity expansion motion is absent. On the assumption of the tendency described hereinabove, in the present embodiment, the presence or absence of the chest cavity expansion motion of the test subject is determined using the difference in the variation period of the component value Q(DC) of the low frequency component between the case in which the chest cavity expansion motion of the test subject is present and the case in which the chest cavity expansion motion is absent.

The control device 26 shown in FIG. 2 is an arithmetic processing device such as a central processing unit (CPU) or a field-programmable gate array (FPGA), and controls the whole of the measurement device 100. The storage device 28 is formed of, for example, a nonvolatile semiconductor memory, and stores a program executed by the control device 26 and a variety of types of data (e.g., a table for identifying the oxygen saturation, and a table for recording the measurement result) used by the control device 26. The control device 26 of the first embodiment executes the program stored in the storage device 28 to thereby realize a calculation section 61 for calculating the oxygen saturation of the test subject, a determination section 63 for determining whether the apnea syndrome of the test subject is OSAS or CSAS, and a counting section 65 for counting the measurement results of the measurement device 100. It should be noted that it is also possible to adopt a configuration of distributing the function of the control device 26 to a plurality of integrated circuits, or a configuration of realizing a part or the whole of the function of the control device 26 with a dedicated electronic circuit. Further, although in FIG. 2, there are illustrated the control device 26 and the storage device 28 as separate elements, it is also possible to realize the control device 26 incorporating the storage device 28 with, for example, an application specific integrated circuit (ASIC).

The calculation section 61 calculates the oxygen saturation of the test subject from the detection signal P generated by the detection device 24. For the identification of the oxygen saturation by the calculation section 61, a known technology can arbitrarily be adopted. For example, it is possible to identify the oxygen saturation using correlation between a variation ratio Φ calculated from the detection signal P and the oxygen saturation. The variation ratio Φ is a ratio of a signal component ratio X2 to a signal component ratio X1 as expressed by the following formula (1). The signal component ratio X1 is an intensity ratio between the component value Q1(AC) of the high frequency component and the component value Q1(DC) of the low frequency component of the first detection signal P1 in the case in which the first light emitting element E1 emits the light L1 (near infrared light). The signal component ratio X2 is an intensity ratio between the component value Q2(AC) of the high frequency component and the component value Q2(DC) of the low frequency component of the second detection signal P2 in the case in which the second light emitting element E2 emits the light L2 (infrared light). The variation ratio Φ of the formula (1) and the oxygen saturation are correlated with each other.

$$Φ = \frac{{X}_{2}}{{X}_{1}} = \frac{{Q}_{2 ( AC )} / {Q}_{2 ( DC )}}{{Q}_{1 ( AC )} / {Q}_{1 ( DC )}} \qquad ( 1 )$$

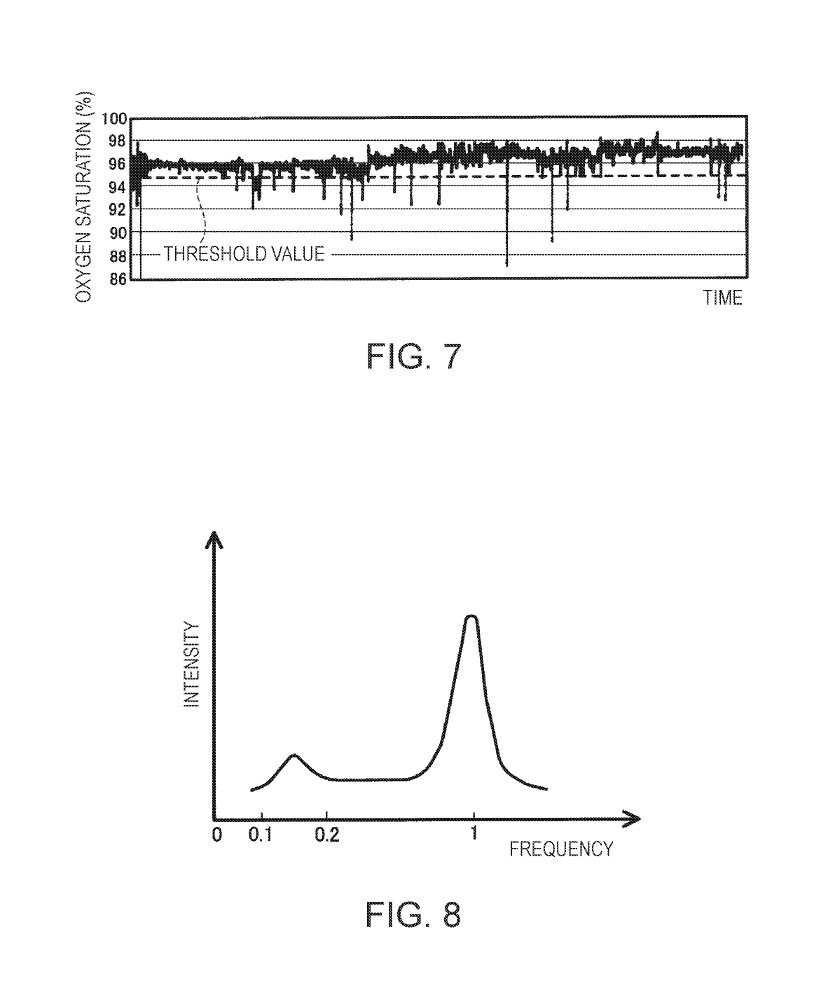

The calculation section 61 calculates the variation ratio Φ from the formula (1) due to the analysis of the first detection signal P1 and the second detection signal P2. Then, the calculation section 61 refers to a table having the numerical values of the variation ratio Φ and the numerical values of the oxygen saturation corresponding to each other to identify the numerical value corresponding to the variation ratio Φ, which is calculated from the first detection signal P1 and the second detection signal P2, as the oxygen saturation (the measurement value) of the test subject. The temporal variation of the oxygen saturation identified by the calculation section 61 is as illustrated in FIG. 7.

The determination section 63 executes the determination on whether or not the test subject is in the apnea syndrome, and the determination on whether the apnea syndrome is OSAS or CSAS. Firstly, the determination section 63 determines whether or not the test subject is in the apnea syndrome. Specifically, the determination section 63 determines whether or not the test subject is in the apnea syndrome in accordance with the result of the determination on whether or not the oxygen saturation calculated by the calculation section 61 has dropped. In the first embodiment, the determination on whether or not the oxygen saturation has dropped is made in accordance with the result of the comparison between the oxygen saturation and a predetermined threshold value. The threshold value is set to a predetermined value (e.g., a numerical value within a range of about 90% through 95%) lower than the oxygen saturation typically observed in the normal respiratory condition. The determination section 63 determines that the test subject is in the apnea syndrome in the case in which the oxygen saturation is lower than the threshold value, and determines that the test subject is in the normal respiratory condition in the case in which the oxygen saturation exceeds the threshold value.

Secondary, in the case in which the determination section 63 has determined that the oxygen saturation is lower than the predetermined threshold value, namely in the case in which the determination section 63 has determined that the test subject is in the apnea syndrome, the determination section 63 determines the presence or absence of the chest cavity expansion motion from the detection signal P generated by the detection device 24. Either one of the first detection signal P1 and the second detection signal P2 used for the calculation of the oxygen saturation is also used for the discrimination between OSAS and CSAS. It should be noted that in the first embodiment, the first detection signal P1 is used. Here, as described above, the detection signal P includes the component value Q(AC) of the high frequency component varying with the period of about 10 seconds in tandem with the pulsation, and the component value Q(DC) of the low frequency component varying with the sufficiently long period compared to the component value Q(AC) of the high frequency component. The component value Q(DC) of the low frequency component in the case in which the chest cavity expansion motion is present varies with the period of about 2 seconds through 10 seconds. Therefore, as illustrated in FIG. 8, in the frequency spectrum of the detection signal P in the case in which the chest cavity expansion motion is present, there are observed a peak in the frequency band no lower than 0.1 Hz and not higher than 0.5 Hz due to the chest cavity expansion motion, and a peak in the vicinity of 1 Hz due to the pulsation. Taking the tendency into consideration, the determination section 63 of the first embodiment determines that the chest cavity expansion motion is present in the case in which the peak is detected in the frequency band no lower than 0.1 Hz and no higher than 0.5 Hz in the frequency spectrum of the first detection signal P1, and determines that the chest cavity expansion motion is absent in the case in which the peak is not detected in this frequency band. Specifically, the determination section 63 converts the first detection signal P1 in the time domain into the frequency spectrum in the frequency domain. For the generation of the frequency spectrum, a known frequency analysis such as short-time Fourier transform is arbitrarily adopted. The determination section 63 determines that the chest cavity expansion motion is present in the case in which the peak is detected in the frequency band no lower than 0.1 Hz and no higher than 0.5 Hz, and determines that the chest cavity expansion motion is absent in the case in which the peak is not detected in that frequency band.

Thirdly, the determination section 63 discriminates the apnea syndrome of the test subject between OSAS and CSAS in accordance with the presence or absence of the chest cavity expansion motion in the case in which the oxygen saturation is lower than the predetermined threshold value. Specifically, in the case in which the oxygen saturation is lower than the predetermined threshold value, the determination section 63 determines that the apnea syndrome is OSAS in the case in which the chest cavity expansion motion is present, and determines that the apnea syndrome is CSAS in the case in which the chest cavity expansion motion is absent.

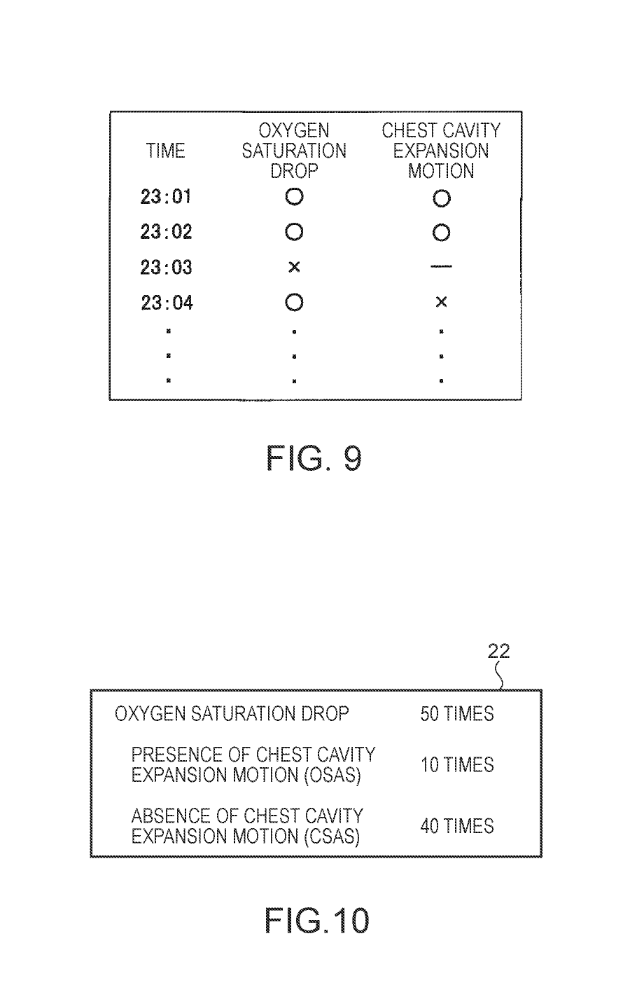

The determination section 63 registers the calculation result of the oxygen saturation and the discrimination result between OSAS and CSAS in the measurement result table shown in FIG. 9 as the measurement result. As illustrated in FIG. 9, in the measurement result table, there are registered the determination result on whether or not the oxygen saturation is lower than the predetermined threshold value, and the determination result on the presence or absence of the chest cavity expansion motion in association with the time of the determination. In the case in which the oxygen saturation is lower than the predetermined threshold value, the determination result on the presence or absence of the chest cavity expansion motion is associated therewith. It should be noted that it is also possible for the determination section 63 to register (i.e., the measurement result is not registered in the case in which the oxygen saturation exceeds the predetermined threshold value) the fact that the oxygen saturation has been lower than the predetermined threshold value and the determination result on the presence or absence of the chest cavity expansion motion only in the case in which the oxygen saturation has been lower than the predetermined threshold value.

The counting section 65 shown in FIG. 2 counts the measurement results. The counting section 65 of the first embodiment counts the number of times (hereinafter referred to as the “number of times of the oxygen saturation drop”) of the drop in the oxygen saturation. Further, the counting section 65 counts the number of times (hereinafter referred to as the “number of times of presence of the chest cavity expansion motion”) of the drop in the oxygen saturation in the state (i.e., OSAS) in which the chest cavity expansion motion is present in the test subject, and the number of times (hereinafter referred to as the “number of times of absence of the chest cavity expansion motion”) of the drop in the oxygen saturation in the state (i.e., CSAS) in which the chest cavity expansion motion is absent in the test subject based on the measurement result table. In the first embodiment, since the presence or absence of the chest cavity expansion motion is determined in the case in which the oxygen saturation drops, the number of times of presence of the chest cavity expansion motion corresponds to the number of times of the case in which the determination section 63 has determined that the chest cavity expansion motion is present, and the number of times of absence of the chest cavity expansion motion corresponds to the number of times of the case in which the determination section 63 has determined that the chest cavity expansion motion is absent. The number of times obtained by adding the number of times of presence of the chest cavity expansion motion and the number of times of absence of the chest cavity expansion motion coincides with the number of times of the oxygen saturation drop. As is understood from the above description, the counting section 65 of the first embodiment is an element for counting the number of times of the drop in the oxygen saturation for each of OSAS and CSAS. The counting of each of the numbers by the counting section 65 is performed in the case in which, for example, a display instruction of the measurement result is provided by the test subject. The counting section 65 makes the display device 22 display the counting result as illustrated in FIG. 10.

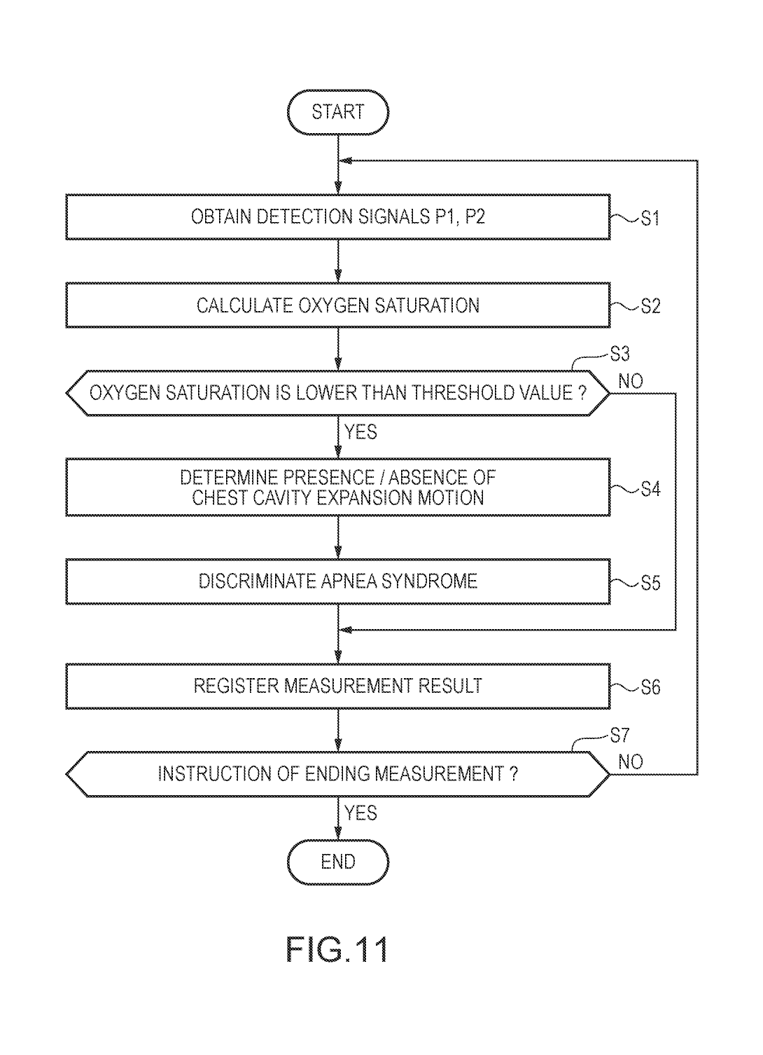

FIG. 11 is a flowchart of the measurement process performed by the control device 26 (the calculation section and the determination section 63). The instruction (start-up of the program) for starting the measurement from the test subject triggers the process shown in FIG. 11.

When the process shown in FIG. 11 is started, the calculation section 61 obtains (S1) the detection signal P (P1 and P2) generated by the detection device 24. The calculation section 61 calculates (S2) the oxygen saturation from the detection signal P thus obtained.

The determination section 63 determines (S3) whether or not the oxygen saturation thus identified is lower than the predetermined threshold value. In the case in which the oxygen saturation is lower than the predetermined threshold value (S3; YES), the determination section 63 determines (S4) the presence or absence of the chest cavity expansion motion based on the first detection signal P1 generated by the detection device 24. The first detection signal P1 is commonly used for both of the calculation of the oxygen saturation in the step S2 and the determination on the presence or absence of the chest cavity expansion motion in the step S4. The determination section 63 discriminates (S5) the apnea syndrome between OSAS and CSAS in accordance with the presence or absence of the chest cavity expansion motion. Specifically, the determination section 63 determines that the apnea syndrome is OSAS in the case in which the chest cavity expansion motion is present, and determines that the apnea syndrome is CSAS in the case in which the chest cavity expansion motion is absent.

The determination section 63 registers (S6) the measurement result to the measurement result table. Specifically, as illustrated in FIG. 9, the determination section 63 registers the determination result on whether or not the oxygen saturation is lower than the predetermined threshold value, and the determination result on the presence or absence of the chest cavity expansion motion in association with the time of the determination. In the case in which the oxygen saturation exceeds the threshold value (S3; NO), the determination (S4) on the presence or absence of the chest cavity expansion motion and the discrimination (S5) of the apnea syndrome are not performed.

In the case in which the instruction for ending the measurement is provided by the test subject (S7; YES), the measurement process is terminated. In contrast, in the case in which the instruction for ending the measurement is not provided (S7; NO), the process from the step S1 to the step S6 is performed in a recurrent manner.

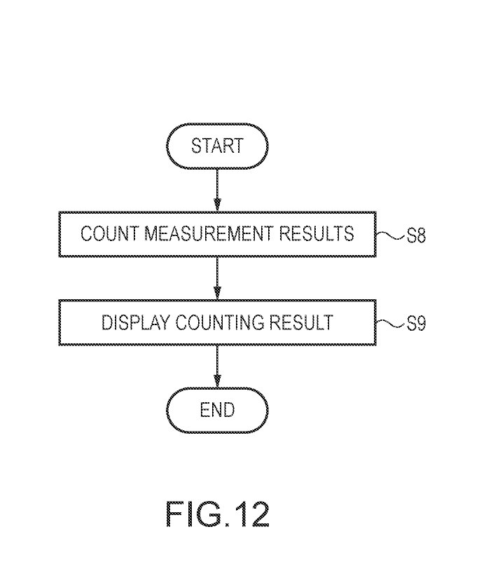

After ending the measurement process described hereinabove, there is performed the counting process for counting the measurement results to display the result. FIG. 12 is a flowchart of the counting process performed by the control device 26 (the counting section 65). The instruction for starting the counting from the test subject triggers the process shown in FIG. 12. It should be noted that the instruction of starting the counting can be made by a third party (e.g., medical personnel such as a medical doctor or a nurse) other than the test subject. When the counting process shown in FIG. 12 starts, the counting section 65 counts (S8) each of the number of times of the oxygen saturation drop, the number of times of presence of the chest cavity expansion motion, and the number of times of absence of the chest cavity expansion motion from the measurement result table. Then, the counting section 65 makes the display device 22 display (S9) the counting result. Specifically, the counting section 65 makes the display device 22 display each of the number of times of the oxygen saturation drop, the number of times of presence of the chest cavity expansion motion, and the number of times of absence of the chest cavity expansion motion as illustrated in FIG. 10.

Here, it is conceivable to adopt a configuration (hereinafter referred to as a “comparative example”) of using a different kind of signal from the pulse wave signal for the determination on the presence or absence of the chest cavity expansion motion of the test subject besides the configuration of the first embodiment, which uses the pulse wave signal (the detection signal P) generated by the detection device 24. In the comparative example, a signal representing the breath sound or the signal representing a body motion, for example, are used for the determination on the presence or absence of the chest cavity expansion motion. Therefore, equipment for determining the presence or absence of the chest cavity expansion motion is necessary separately from the equipment for generating the pulse wave signal for calculating the oxygen saturation. In contrast to the comparative example, in the first embodiment, the detection signal P is commonly used for both of the calculation of the oxygen saturation and the determination on the presence or absence of the chest cavity expansion motion. Therefore, the equipment for generating the different kind of signal from the pulse wave signal is unnecessary. Therefore, the configuration of the measurement device 100 is simplified (by extension, miniaturized) compared to the comparative example.

Further, in the first embodiment, the number of times of the oxygen saturation drop is counted for each of OSAS and CSAS. Therefore, compared to the configuration of counting the number of times of the oxygen saturation drop without discriminating between the OSAS and CSAS, it is possible to individually figure out the tendency (e.g., the frequency) that the oxygen saturation becomes lower than the predetermined threshold value for each of OSAS and CSAS.

### Second Embodiment

A second embodiment of the invention will be described. It should be noted that in each of the configurations illustrated hereinafter, regarding the elements substantially the same in operation and function as those in the first embodiment, the symbols used in the description of the first embodiment are diverted, and the detailed description of each of such elements are arbitrarily omitted.

The measurement device 100 according to the first embodiment performs the determination on the presence or absence of the apnea syndrome, and the determination on whether the apnea syndrome is OSAS or CSAS. The measurement device 100 according to the second embodiment identifies the sleep depth of the test subject in addition to performing the determination on the presence or absence of the apnea syndrome, and the determination on whether the apnea syndrome is OSAS or CSAS similarly to the first embodiment. The sleep depth denotes an index representing the depth of sleep of the test subject. The measurement device 100 according to the second embodiment identifies either one of the stage 1 through stage 4 as a level of the sleep depth. The sleep becomes deeper in the order of stage 1, stage 2, stage 3, and stage 4. In other words, the stage 1 represents the shallowest sleep state, and the stage 4 represents the deepest sleep state. Normally, the shallower the sleep of the test subject is, the larger the body motion (an amount of the motion and a frequency of the motion) such as a roll-over becomes. On the assumption of the tendency described hereinabove, the measurement device 100 according to the second embodiment detects the body motion of the test subject to identify the sleep depth of the test subject.

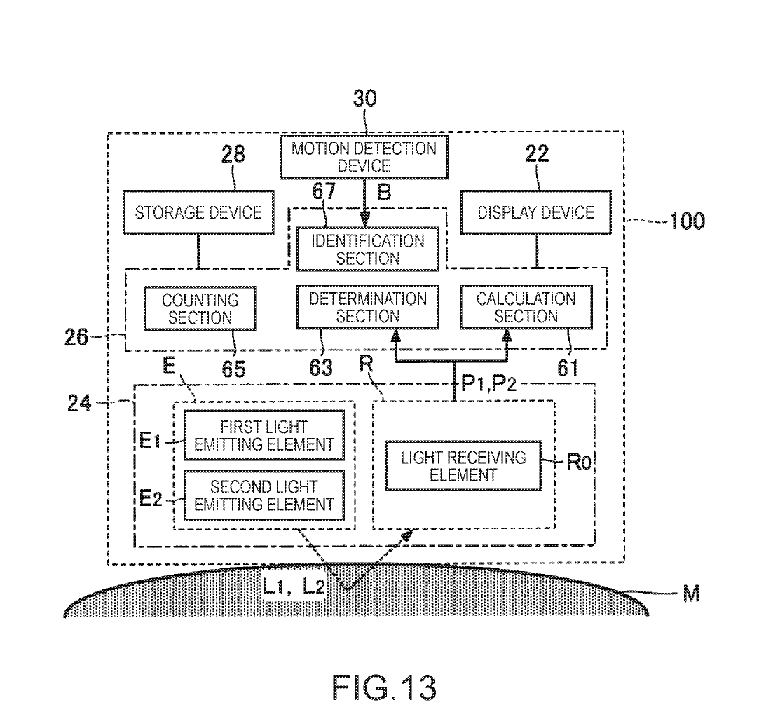

FIG. 13 is a configuration diagram focusing attention on the function of the measurement device 100 according to a second embodiment. The measurement device 100 according to the second embodiment is provided with a motion detection device 30 in addition to including the display device 22, the detection device 24, the control device 26, and the storage device 28 similarly to the first embodiment. The motion detection device 30 is a body motion sensor for detecting the body motion of the test subject. For example, an acceleration sensor for detecting the orthogonal three axes acceleration is preferably used as the motion detection device 30. Specifically, the motion detection device 30 generates a body motion signal B in accordance with the body motion of the test subject.

The control device 26 of the second embodiment executes the program stored in the storage device 28 to thereby realize the calculation section 61 for calculating the oxygen saturation of the test subject, the determination section 63 for determining whether the apnea syndrome of the test subject is OSAS or CSAS, a counting section 65 for counting the measurement results of the measurement device 100, and an identification section 67 for identifying the sleep depth. The calculation section 61 calculates the oxygen saturation similarly to the first embodiment. The determination section 63 executes the determination (i.e., the determination on whether or not the oxygen saturation is lower than the predetermined threshold value) on whether or not the test subject is in the apnea syndrome, and the discrimination of the apnea syndrome between OSAS and CSAS similarly to the first embodiment.

In the case in which the determination 63 has determined that the oxygen saturation is lower than the predetermined threshold value, the identification section 67 identifies the sleep depth of the test subject from the body motion signal B generated by the motion detection device 30. Specifically, the identification section 67 of the second embodiment calculates the displacement of the body of the test subject due to the integration of the body motion signal B, and then identifies either one of the stages 1 through 4 as the sleep depth in accordance with the displacement amount per unit time. Specifically, the identification section 67 identifies the stage in a range, to which the displacement thus calculated corresponds, as the sleep depth out of a plurality of ranges of the displacement amount prepared in advance respectively for the stages 1 through 4.

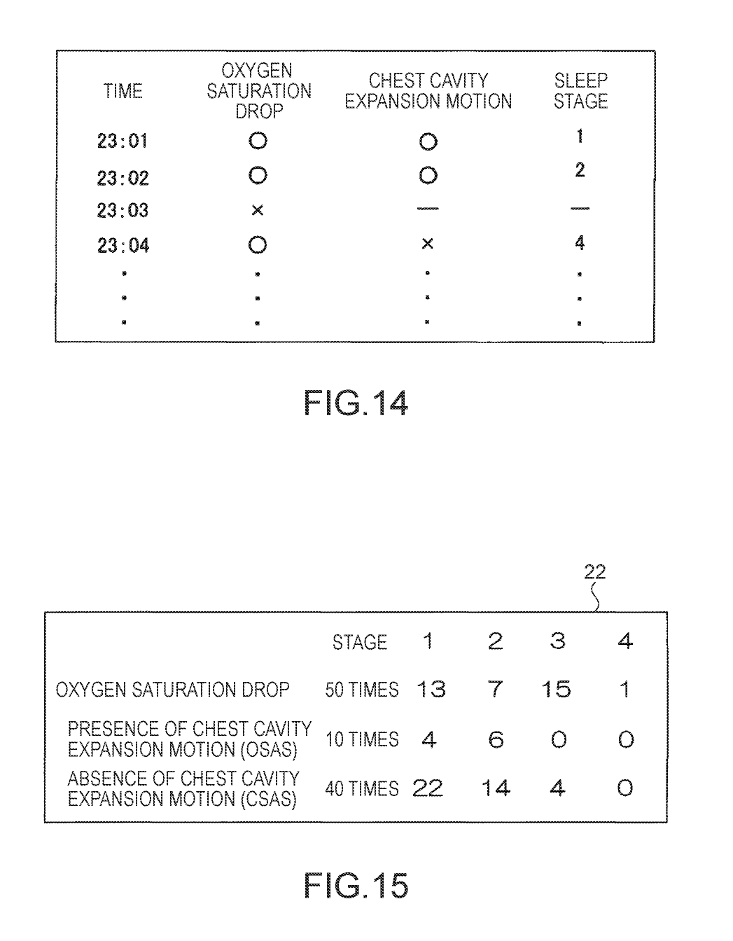

The determination section 63 registers the measurement result to the measurement result table shown in FIG. 14. The determination section 63 according to the second embodiment registers the sleep depth in the measurement result table as the measurement result in addition to the calculation result of the oxygen saturation and the discrimination result between OSAS and CSAS. As illustrated in FIG. 14, the calculation result of the oxygen saturation and the discrimination result between OSAS and CSAS are registered in the measurement result table similarly to the first embodiment. As illustrate in FIG. 14, the sleep depth is registered in the measurement result table in association with the calculation result of the oxygen saturation in the case in which the oxygen saturation is lower than the predetermined threshold value.

The counting section 65 shown in FIG. 13 counts the number of times of the oxygen saturation drop in each of the levels of the sleep depth. Specifically, in the case in which the display instruction of the measurement result is provided from the test subject, the counting section 65 counts the number of times of the oxygen saturation drop for each of the levels of the sleep depth from the measurement result table, and at the same time, also counts the number of times of presence of the chest cavity expansion motion and the number of times of absence of the chest cavity expansion motion for each of the levels of the sleep depth. The counting section 65 makes the display device 22 display the counting result as illustrated in FIG. 13.

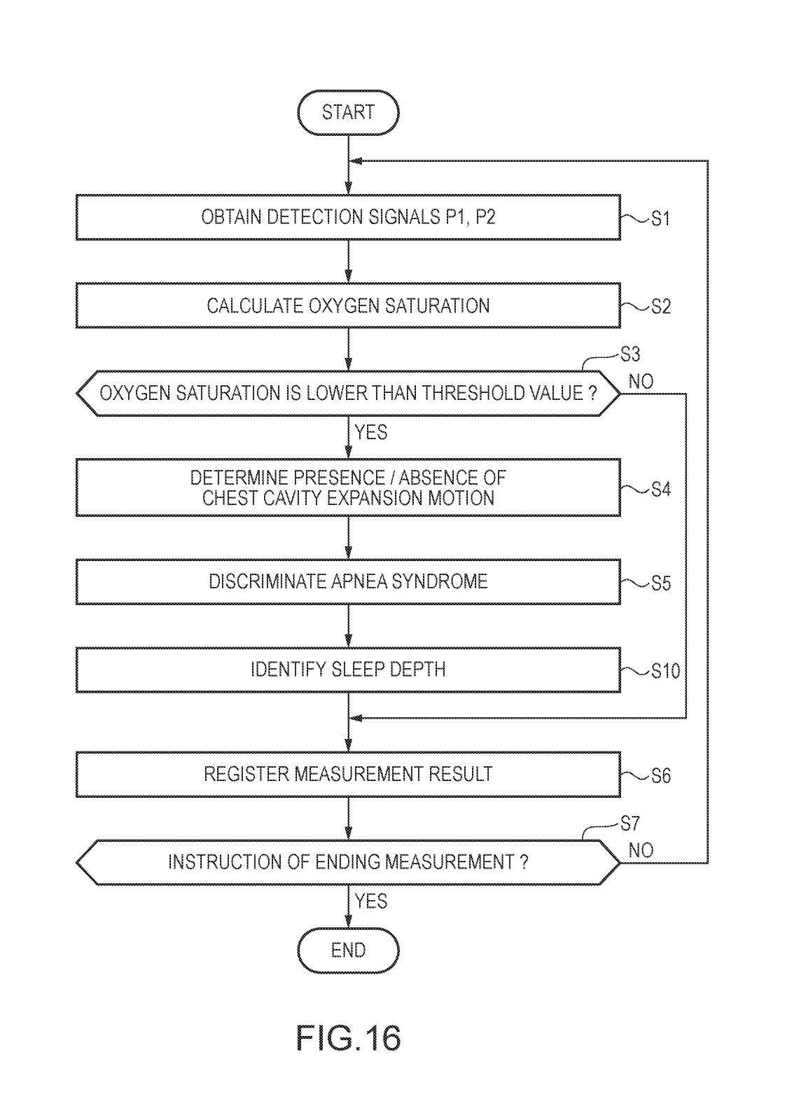

FIG. 16 is a flowchart of the measurement process performed by the control device 26 (the calculation section 61, the determination section 63, and the identification section 67) in the second embodiment. From the start of the process to the process of discriminating between OSAS and CSAS (S1 through S5) is substantially the same as in the first embodiment. After the determination section 63 discriminates between OSAS and CSAS in the step S5, the identification section 67 identifies (S10) the sleep depth in accordance with the body motion signal B generated by the motion detection device 30. The determination section 63 registers (S6) the measurement result to the measurement result table. Specifically, the determination section 63 registers the calculation result of the oxygen saturation, the discrimination result between OSAS and CSAS, and the sleep depth in the measurement result table shown in FIG. 14 as the measurement result. In the case in which the oxygen saturation exceeds the threshold value (S3; NO), the determination (S4) on the presence or absence of the chest cavity expansion motion, the discrimination (S5) of the apnea syndrome, and the identification (S10) of the sleep depth are not performed.

In the case in which the instruction for ending the measurement is provided from the test subject or the medical personnel to the counting section 65 (S7; YES), the measurement process is terminated. In contrast, in the case in which the instruction for ending the measurement is not provided (S7; NO), the process from the step S1 to the step S6 is performed in a recurrent manner.

Similarly to the first embodiment, the instruction from the test subject or the medical personnel triggers the counting process shown in FIG. 12. When the counting process shown in FIG. 12 starts, the counting section 65 counts (S8) each of the number of times of the oxygen saturation drop, the number of times of presence of the chest cavity expansion motion, and the number of times of absence of the chest cavity expansion motion for each of the levels of the sleep depth from the measurement result table. The counting section 65 makes the display device 22 display (S9) the counting result. Specifically, as illustrated in FIG. 15, the counting section 65 makes the display device 22 display each of the number of times of the oxygen saturation drop, the number of times of presence of the chest cavity expansion motion, and the number of times of absence of the chest cavity expansion motion for each of the levels of the sleep depth.

As is understood from the above description, substantially the same advantages as in the first embodiment can also be obtained in the second embodiment. In particular in the second embodiment, the number of times of the oxygen saturation drop is counted for each of the levels of the sleep depth. Therefore, compared to the configuration of counting the number of times of the oxygen saturation drop without discriminating between the levels of the sleep depth, it is possible to figure out the tendency (e.g., the frequency) that the oxygen saturation becomes lower than the predetermined threshold value for each of the levels of the sleep depth.

### MODIFIED EXAMPLES

Each of the embodiments described hereinabove can variously be modified. Specific modified configurations will hereinafter be illustrated. It is also possible to arbitrarily combine two or more configurations arbitrarily selected from the following illustrations.

(1) In each of the embodiments described above, whether or not the oxygen saturation is lower than the predetermined threshold value is determined, but the method of determining whether or not the oxygen saturation drops is not limited to the above illustrations. For example, it is also possible to determine that the oxygen saturation drops in the case in which, for example, the amount of the drop in the oxygen saturation exceeds a predetermined threshold value. In other words, both of the fact that the oxygen saturation exceeds a predetermined threshold value and the fact that the amount of the drop in the oxygen saturation exceeds a predetermined threshold value can comprehensively be expressed as the “oxygen saturation drop.”

(2) In each of the embodiments described above, the determination section 63 performs the determination on the presence or absence of the chest cavity expansion motion in the case in which the oxygen saturation has dropped, but can also perform the determination on the presence or absence of the chest cavity expansion motion irrespective of the presence or absence of the drop of the oxygen saturation. Therefore, the determination section 63 can comprehensively be expressed as an element for determining the presence or absence of the chest cavity expansion motion of the test subject from the detection signal P. It should be noted that in each of the embodiments described above for determining the presence or absence of the chest cavity expansion motion in the case in which the oxygen saturation drops, the processing load of the measurement device 100 is reduced compared to the configuration of determining the presence or absence of the chest cavity expansion motion irrespective of the presence or absence of the drop in the oxygen saturation. Further, the identification section 67 identifies the sleep depth in the case in which the determination section 63 has determined that the oxygen saturation has dropped in the second embodiment, but can also identify the sleep depth irrespective of the presence or absence of the drop of the oxygen saturation. Therefore, the identification section 67 can comprehensively be expressed as an element for identifying the sleep depth of the test subject.

(3) The determination section 63 determines the presence or absence of the chest cavity expansion motion in accordance with the presence or absence of the peak in the frequency band no lower than 0.1 Hz and no higher than 0.5 Hz out of the frequency spectrum of the detection signal P in each of the embodiments described above, but can also determine the presence or absence of the chest cavity expansion motion in accordance with the variation period of the component value Q(DC) of the low frequency component of the detection signal P. In other words, the process in the frequency domain can be omitted. Specifically, the determination section 63 determines that the chest cavity expansion motion is present in the case in which the variation period of the component value Q(DC) of the low frequency component out of the detection signal P (P1 or P2) is within a predetermined range, and determines that the chest cavity expansion motion is absent in the case in which the variation period is not within the predetermined range. As described above, the component value Q(DC) of the low frequency component has a tendency of varying with the period no shorter than 2 seconds and not longer than 10 seconds in the case in which the chest cavity expansion motion is present. Therefore, the predetermined range is preferably set to the range no shorter than 2 seconds and not longer than 10 seconds. According to the configuration described above for determining the presence or absence of the chest cavity expansion motion in accordance with the variation period of the component value Q(DC) of the low frequency component of the detection signal P, it is possible to accurately determine the presence or absence of the chest cavity expansion motion using the tendency that the component value Q(DC) of the low frequency component of the detection signal P varies with the period no shorter than 2 seconds and not longer than 10 seconds in the case in which the chest cavity expansion motion is present. In contrast, according to each of the embodiments described above for determining the presence or absence of the chest cavity expansion motion in accordance with the presence or absence of the peak in the frequency band no lower than 0.1 Hz and no higher than 0.5 Hz out of the frequency spectrum of the detection signal P, it is possible to accurately determine the presence or absence of the chest cavity expansion motion using the tendency that the variation period is different between the case in which the chest cavity expansion motion is present and the case in which the chest cavity expansion motion is absent.

(4) In each of the embodiments described above, the determination section 63 discriminates between OSAS and CSAS in accordance with the presence or absence of the chest cavity expansion motion in the case in which the oxygen saturation drops, but it is not essential for the determination section 63 to discriminate the apnea syndrome between OSAS and CSAS. The determination section 63 can comprehensively be expressed as an element for determining the presence or absence of the chest cavity expansion motion of the test subject from the detection signal P. It should be noted that in each of the embodiments for discriminating between the OSAS and CSAS in accordance with the presence or absence of the chest cavity expansion motion in the case in which the oxygen saturation drops, it is possible to discriminate between OSAS and CSAS in accordance with the combination of the presence or absence of the oxygen saturation drop and the presence or absence of the chest cavity expansion motion.

(5) In each of the embodiments described above, the measurement device 100 counts the number of times of the oxygen saturation drop, but it is not essential for the measurement device 100 to count (i.e., the counting section 65) the number of times of the oxygen saturation drop. It should be noted that according to each of the embodiments described above for counting the number of times of the oxygen saturation drop, it is possible to figure out the tendency (e.g., the frequency) of the oxygen saturation drop.

(6) In the second embodiment, the acceleration sensor for detecting the acceleration is used as the motion detection device 30, but it is also possible to use a vibration sensor for detecting a vibration or an ultrasonic sensor as the motion detection device 30.

(7) In each of the embodiments described above, the oxygen saturation is identified referring to the table having the numerical values of the oxygen saturation and the numerical values of the variation ratio Φ calculated by the formula (1) in association with each other, but it is also possible to calculate the oxygen saturation using a predetermined arithmetic expression describing the relationship between the variation ratio Φ and the oxygen saturation.

(8) In each of the embodiments described above, the light L1 emitted by the first light emitting element and the light L2 emitted by the second light emitting element E2 are commonly received by the light receiving element R0, but can also be received by respective light receiving elements R1, R2 separate from each other. The light receiving element R1 receives the light L1 emitted from the first light emitting element E1 and then transmitted through the measurement region M to generate the first detection signal P1 corresponding to the light reception level. The light receiving element R2 receives the light L2 emitted from the second light emitting element E2 and then transmitted through the measurement region M to generate the second detection signal P2 corresponding to the light reception level.

(9) In the embodiments described above, the measurement device 100, as a single unit, performs the calculation of the oxygen saturation, the determination of the apnea syndrome, and the display of the measurement value, but it is also possible to realize the functions of the measurement device 100 illustrated in each of the embodiments described above with a plurality of devices. For example, it is also possible to realize the calculation of the oxygen saturation, the determination of the apnea syndrome, and the display of the measurement result using the detection device 24 and a terminal device (e.g., a cellular phone or a smartphone) capable of communicating with the detection device 24 as the measurement device 100. Specifically, the first detection signal P1 and the second detection signal P2 generated by the detection device 24 are transmitted to the terminal device. The terminal device evaluates the apnea syndrome based on the first detection signal P1 and the second detection signal P2 received from the detection device 24, and makes the display device of the terminal device display the measurement result. As is understood from the illustrations described above, it is also possible to configure the detection device 24 and the control device 26 so as to be separate from each other. It is also possible to make the display device as a separate body from the measurement device 100 display the measurement result. Further, it is also possible to adopt a configuration (e.g., a configuration realized by an application program executed by, for example, a terminal device) in which one or more of the calculation section 61, the determination section 63, and the counting section 65 are provided to the terminal device. As is understood from the above description, the measurement device 100 can also be realized by a plurality of devices configured separately from each other.

(10) In the embodiments described above, there is illustrated the measurement device 100 formed of the belt 14 and the housing section 12, but the specific configuration of the measurement device 100 is arbitrary. It is possible to adopt the measurement device 100 with an arbitrary configuration such as a patch type which can be pasted on the body of the test subject, an earring type which can be attached to an ear lobe of the test subject, a finger-mount type (e.g., a nail-mount type) which can be mounted to the fingertip of the test subject, or a head-mount type which can be mounted on the head of the test subject. It should be noted that in the state of mounting the measurement device 100 such as the finger-mount type, it is supposed that there is a possibility that there is posed a problem for the daily life. Therefore, from the viewpoint of generating the detection signal P on a steady basis without posing a problem for the daily life, the measurement device 100 of the configuration described above which can be mounted on the wrist of the test subject with the belt 14 is particularly preferable. It should be noted that it is also possible to realize the measurement device 100 having a configuration to be mounted on (e.g., externally attached to) a variety of types of electronic apparatus such as a wristwatch.

(11) The invention can be identified as an operation method (a measurement method) of the measurement device 100. Specifically, in the measurement method as a preferred embodiment of the invention, a computer calculates the oxygen saturation from the detection signal of the test subject, and then determines the presence or absence of the chest cavity expansion motion of the test subject from the detection signal P.

The entire disclosure of Japanese Patent Application No. 2016-186937 is hereby incorporated herein by reference.

What is claimed is:

1. A measurement device comprising:
a calculation section adapted to calculate oxygen saturation from a pulse wave signal of a test subject; and
a determination section adapted to determine presence or absence of chest cavity expansion motion of the test subject from the pulse wave signal.

2. The measurement device according to claim 1, wherein
the determination section determines the presence or absence of the chest cavity expansion motion in a case in which the oxygen saturation drops.

3. The measurement device according to claim 1, wherein
the determination section determines that the chest cavity expansion motion is present in a case in which a peak is detected in a frequency band no lower than 0.1 Hz and no higher than 0.5 Hz out of frequency spectrum of the pulse wave signal, and determines that the chest cavity expansion motion is absent in a case in which no peak is detected in the frequency band no lower than 0.1 Hz and no higher than 0.5 Hz.

4. The measurement device according to claim 1, wherein
the determination section determines that the chest cavity expansion motion is present in a case in which a variation period of a component value of a low frequency component of the pulse wave signal is within a predetermined range, and determines that the chest cavity expansion motion is absent in a case in which the variation period of the component value of the low frequency component of the pulse wave signal is out of the predetermined range.

5. The measurement device according to claim 4, wherein
the predetermined range is a range no shorter than 2 seconds and not longer than 10 seconds.

6. The measurement device according to claim 1, wherein
in a case in which oxygen saturation drops, the determination section determines that an obstructive sleep apnea syndrome occurs in a case in which it is determined that the chest cavity expansion motion is present, and determines that a central sleep apnea syndrome occurs in a case in which it is determined that the chest cavity expansion motion is absent.

7. The measurement device according to claim 1, further comprising:
a counting section adapted to count a number of times of the oxygen saturation drop.

8. The measurement device according to claim 7, wherein
the counting section counts the number of times of the oxygen saturation drop for each of the obstructive sleep apnea syndrome and the central sleep apnea syndrome.

9. The measurement device according to claim 7, further comprising:
an identification section adapted to identify sleep depth of the test subject,
wherein the counting section counts the number of times of the oxygen saturation drop in each of levels of the sleep depth.

10. A measurement method comprising:
calculating, by a computer, oxygen saturation from a pulse wave signal of a test subject; and
determining, by the computer, presence or absence of a chest cavity expansion motion of the test subject.
